(19) United States
(12) Patent Application Publication
(10) Pub. No.: US 2007/0038228 A1
(43) Pub. Date: Feb. 15, 2007

(54) Depilation device with a depilation tape

(75) Inventors: Adolf Ramusch, Schiefling am See (AT); Rudolf Wurmitzer, Pischeldorf (AT); Josef Erlacher, St. Salvator (AT); Doris Koefer, Klagenfurt (AT)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V. GROENEWOUDSEWEG 1, 5621 BA EINDHOVEN (NL)

(21) Appl. No.: 10/552,768

(22) PCT Filed: Apr. 14, 2004
(86) PCT No.: PCT/IB04/50437

§ 371 Date: Oct. 12, 2005

(30) Foreign Application Priority Data
Apr. 17, 2003 (EP) 03 101 071.3

(51) Int. Cl. A61B 17/50

(52) U.S. Cl. 606/133

(57) ABSTRACT

In a depilation device (1) with a reel (16) for accommodating a tape roll (15) formed by winding up of a depilation tape (2), said reel (16) advantageously performs a dual function, such that the reel (16) is designed both for un-reeling the depilation tape (2) from the tape roll (15) accommodated on the reel (16) and for winding up the depilation tape (2) onto the tape roll (15) present on the reel (16).

The invention relates to a depilation device comprising a housing and a reel said reel being supported in the depilation device so as to be rotatable about a reel axis and being provided and constructed for accommodating a tape roll formed by winding up of a depilation tape.

A depilation device as described in the first paragraph above is known from the patent document EP 0 738 481 B1. The known depilation device comprises a housing that can be held in one hand. An accommodation chamber is provided in the housing. A reel formed without flange is rotatably arranged in the accommodation chamber. The reel is located for the major portion in the accommodation chamber and accordingly inside the housing of the depilation device. The reel projects from the accommodation chamber into the exterior only with a reel portion having the shape of a cylinder segment. The reel can be driven by a motor of the known depilation device which is accommodated in the housing, a gear transmission being provided as a driving means between the motor and the reel. The reel in the known depilation device is designed only for winding up a depilation tape that was previously applied to the skin of a person. The known depilation device, however, is not provided and constructed for applying the depilation tape to the skin of a person, so that the application of the depilation tape to the skin of a person requires a procedure independent of the known depilation device or a means independent of the known depilation device.

The invention has for its object to eliminate the condition described above and to provide an improved depilation device.

To achieve the above object, inventive features are provided in a depilation device according to the invention such that a depilation device according to the invention can be characterized in the following manner:

Depilation device comprising a housing and a reel said reel being supported in the depilation device so as to be rotatable about a reel axis and being provided and constructed for accommodating a tape roll formed by winding up of a depilation tape, wherein the reel performs a dual function and is designed both for unreeling the depilation tape from the tape roll present on the reel so as to apply a portion of the depilation tape to the skin of a person, and also for winding up the depilation tape onto the tape roll present on the reel so as to pull off a portion of the depilation tape previously applied to the skin of a person from the skin of this person again, and wherein the reel can be driven into rotation by drive means during winding up of the depilation tape, and wherein the reel can be driven into rotation by the depilation tape during unreeling of the depilation tape without being hampered by the drive means.

The provision of the features according to the invention achieves in a simple manner and with only a small additional expenditure that a depilation device according to the invention is equipped for and can be used for unreeling the depilation tape as well as for winding up the depilation tape, while nevertheless only one reel can suffice in a particularly advantageous manner because the one reel performs a dual function and is used both for unreeling the depilation tape and for winding up the depilation tape. During operation of the depilation tape according to the invention, a portion of the depilation tape can be applied to the skin of a person a few times in succession and be pulled from the skin of this person again, during which each time an unreeling and a winding of the depilation tape take place in succession, which involves the advantage of a multiple use of a portion of the depilation tape and accordingly the use of a comparatively small quantity of depilation tape.

The drive means in a depilation device according to the invention may comprise a spring motor or a return spring device. It was found to be particularly advantageous, however, if the drive means comprise an electric motor. This is advantageous for as even as possible a drive of the depilation tape and accordingly a continuous removal of the depilation tape from the skin of a person.

It was found to be particularly advantageous in a depilation device according to the invention with an electric motor if in addition the characteristics of claim 3 are provided. Such a construction is advantageous because an interference with the unreeling of the depilation tape by the drive means can be prevented in a particularly simple manner.

It was found to be particularly advantageous in a depilation device according to the invention if in addition the characteristics of claim 4 are provided. This advantageously achieves that a depilation tape with a hair removal medium capable of softening by heating can be used in a depilation device according to the invention, while it is safeguarded that no renewed heating of the depilation tape takes place during winding up of a depilation tape that was previously applied to the skin of a person, so that no undesirable sticking together of the newly wound depilation tape takes place. It is achieved thereby, furthermore, that the heating device does not exert a friction load on the depilation tape to be wound during winding up of a portion of the depilation tape that was previously provided on the skin of a person, which is advantageous for minimizing the load on the drive means.

It was found to be particularly advantageous in a depilation device according to the invention with a heating device that is adjustably mounted if in addition the characteristics of claim 5 are provided. It is advantageously achieved thereby that the lifting of the application roller from the skin of a person automatically moves the heating device into its rest position, so that an undesirable heating of the depilation tape removed from the skin of a person and wound up on the reel again is prevented in a particularly simple and reliable manner. The adjustment of the application roller into its rest position, however, may alternatively be achieved by means of a button or a direct gripping and moving by means of two fingers of a hand.

The above and further aspects of the invention will become apparent from the following description of embodiments and are explained in more detail therein with reference to these embodiments.

The invention will be explained in more detail below with reference to embodiments shown in the drawings, however, the invention is by no means limited thereto.

FIG. 1 is a bottom view of a depilation device in a first embodiment of the invention.

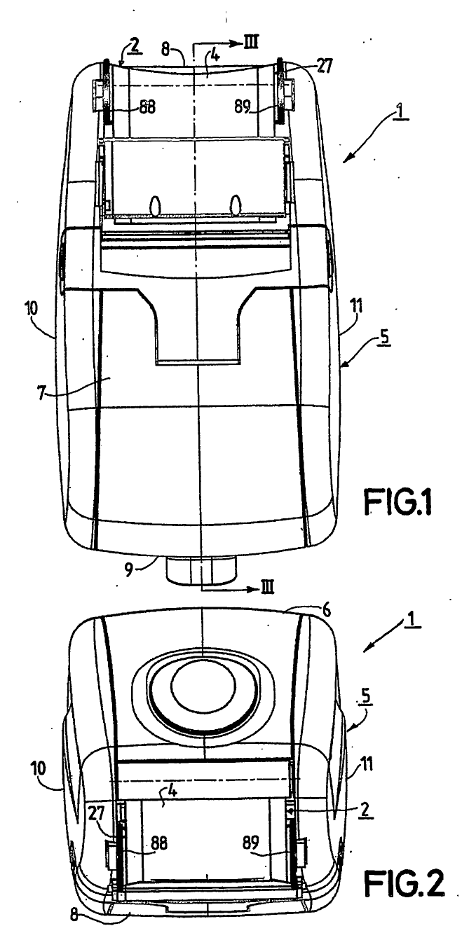

FIG. 2 is a front elevation of the depilation device of FIG. 1.

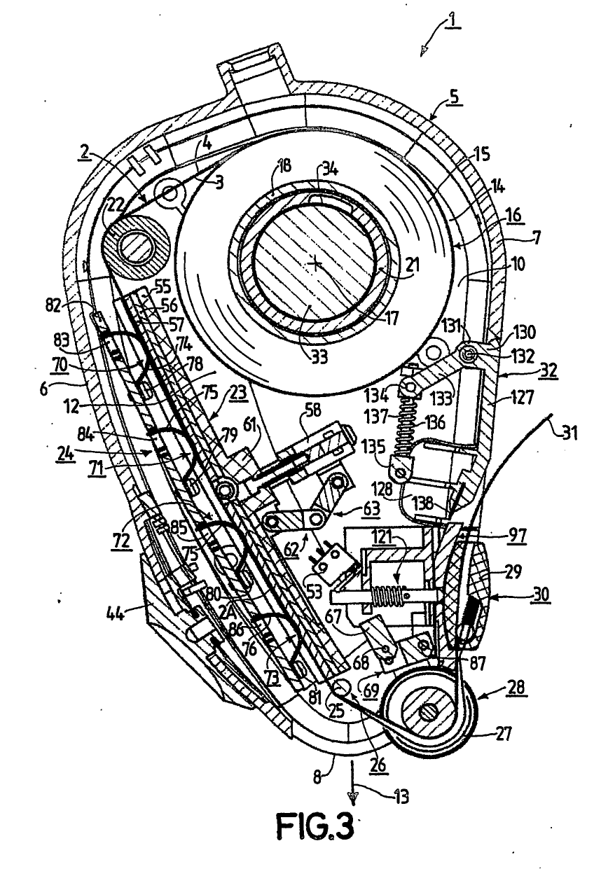

FIG. 3 shows the depilation device of FIGS. 1 and 2 in a cross-section taken on the line III-III in FIG. 1 and on a larger scale.

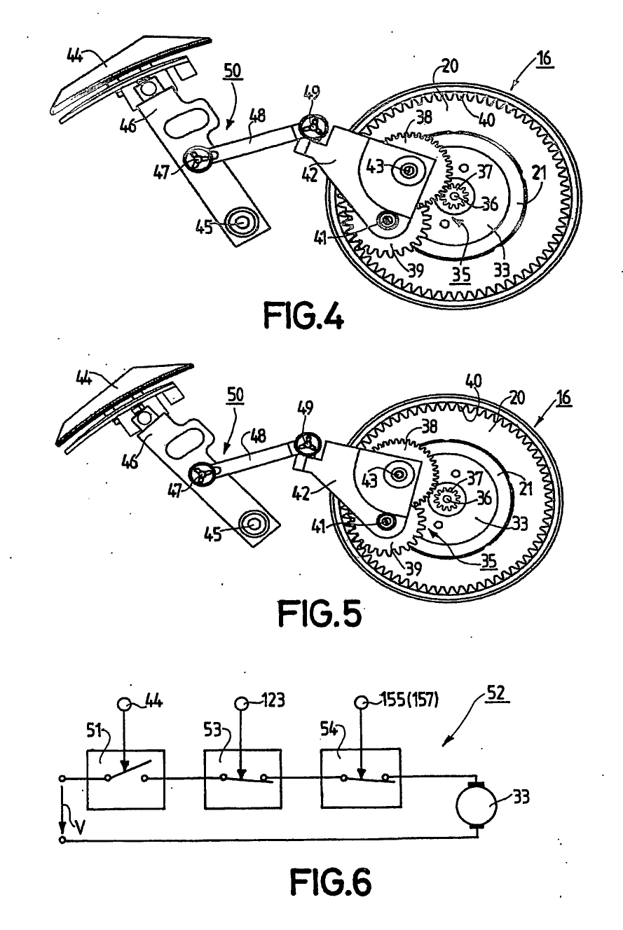

FIG. 4 is a plan view of part of the depilation device of FIGS. 1 to 3, which part of the depilation device comprises an ON/OFF slider button which occupies its ON position in FIG. 4.

FIG. 5 shows in the same manner as FIG. 4 the part of the depilation device of FIG. 4 with the ON/OFF slider button occupying its OFF position.

FIG. 6 is a diagrammatic block switching diagram of motor control means for the motor of the depilation device of FIGS. 1 to 3.

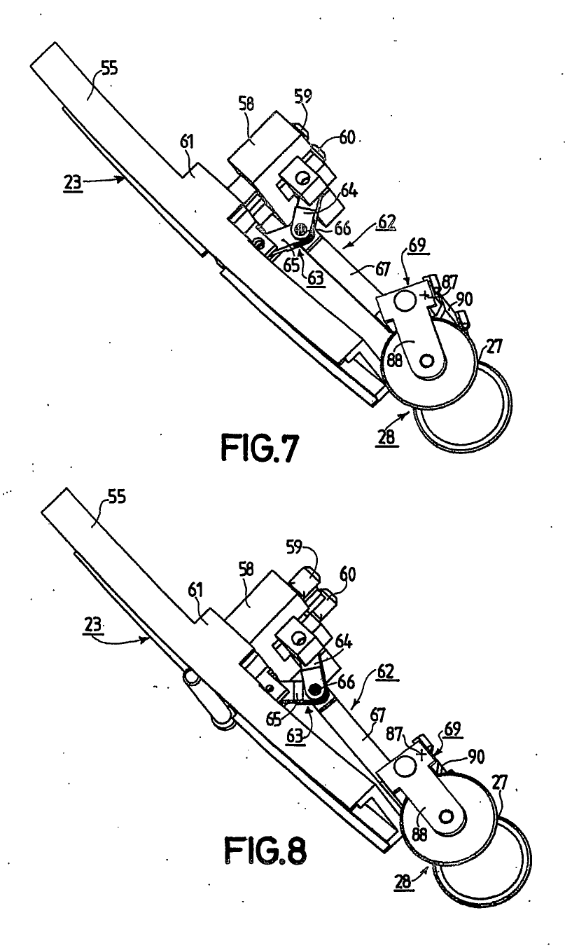

FIG. 7 is an oblique view of a further part of the depilation device of FIGS. 1 to 3, which further part comprises an adjustable heating device here occupying its operational position.

FIG. 8 shows in the same manner as FIG. 7 the further part of the depilation device shown in FIG. 7, with the heating device occupying its rest position.

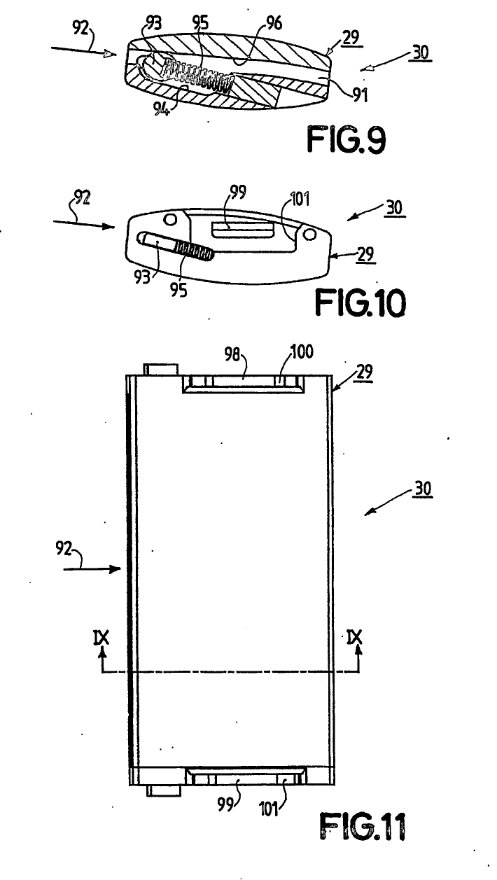

FIG. 9 is a cross-section taken on the line IX-IX in FIG. 11 of a stop block of the depilation device which is connected to the depilation tape in accordance with FIG. 3.

FIG. 10 is a side elevation of the stop block of FIG. 9.

FIG. 11 is a plan view of the stop block of FIGS. 9 and 10.

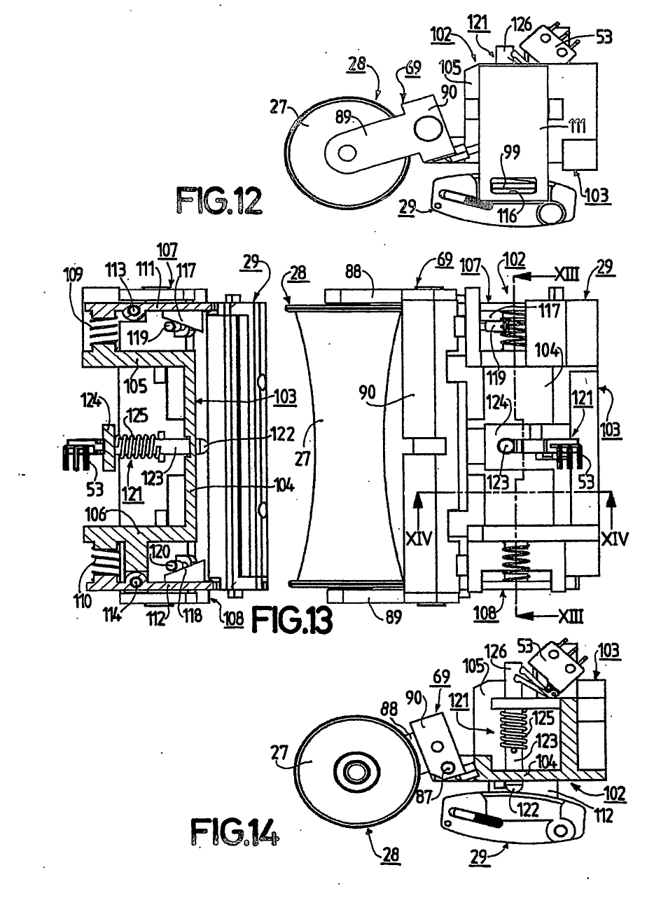

FIG. 12 is a side elevation of a further part of the depilation device, which part of the depilation device comprises an application roller, a stop block, and retaining means for the stop block, the application roller being shown in its rest position.

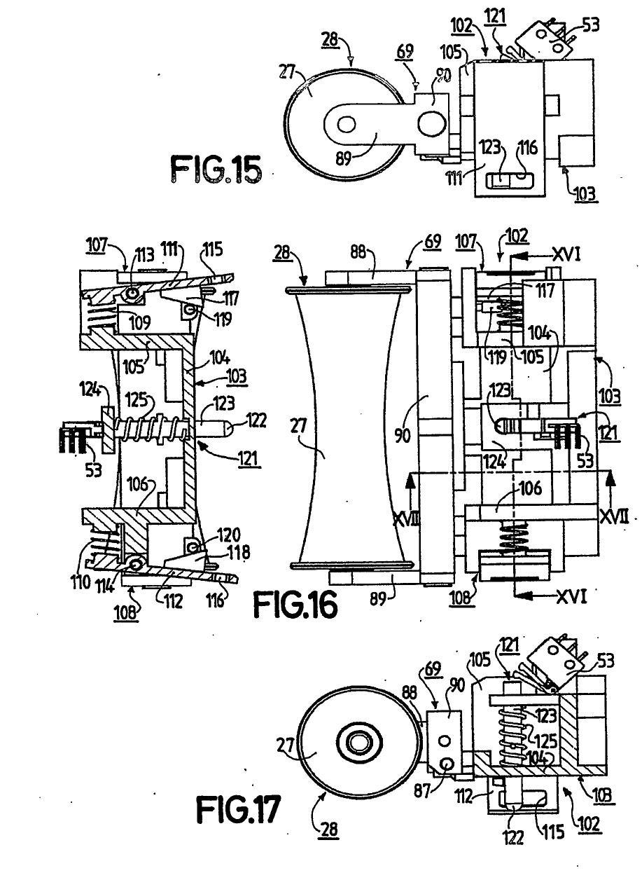

FIG. 13 shows the further part of FIG. 12 in a plan view and in a cross-section taken on the line XIII-XIII in FIG. 13.

FIG. 14 shows the further part of FIGS. 12 and 13 in a cross-section taken on the line XIV-XIV in FIG. 10.

FIGS. 15, 16, and 17 also show the further part of FIGS. 12, 13, and 14, but the application roller is shown in its operational position in FIGS. 15, 16, and 17.

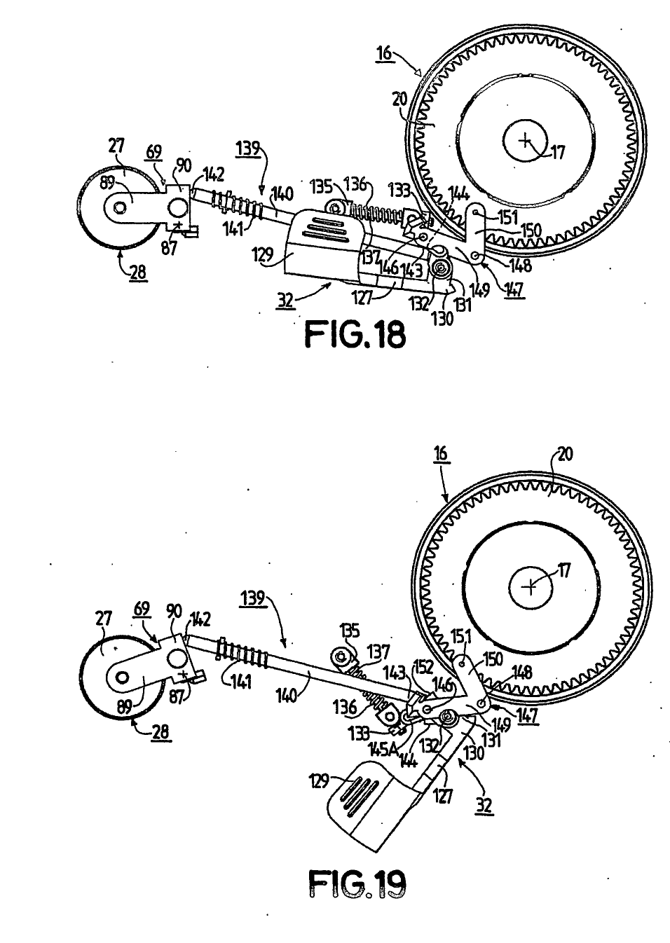

FIG. 18 is a side elevation of a further part of the depilation device of FIGS. 1 to 3, which further part comprises the application roller and the reel that is to co-operate with the depilation tape, the application roller being shown in its operational position.

FIG. 19 shows in a similar manner as FIG. 18 the further part of FIG. 18, with the application roller in its rest position.

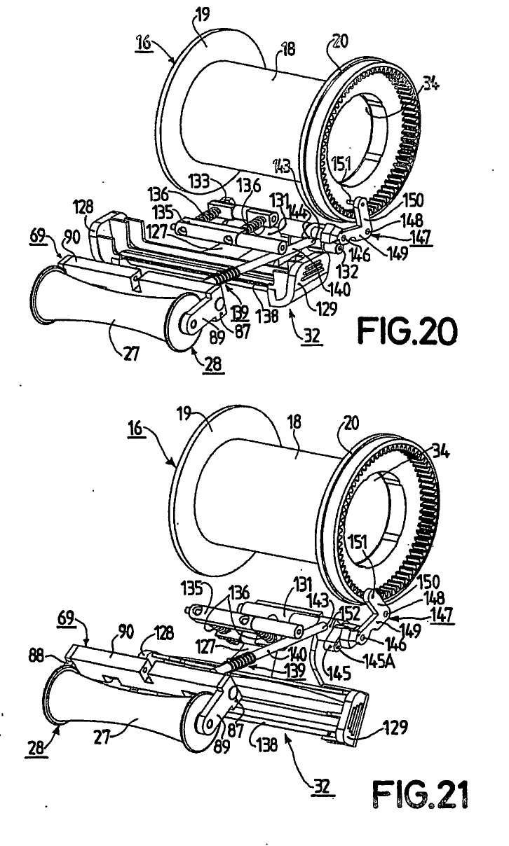

FIG. 20 is an oblique view of the further part of FIGS. 18 and 19 of the depilation device of FIGS. 1 to 3, with the application roller in its operational position.

FIG. 21 shows in a similar manner as FIG. 20 the further part of FIG. 20, with the application roller in its rest position.

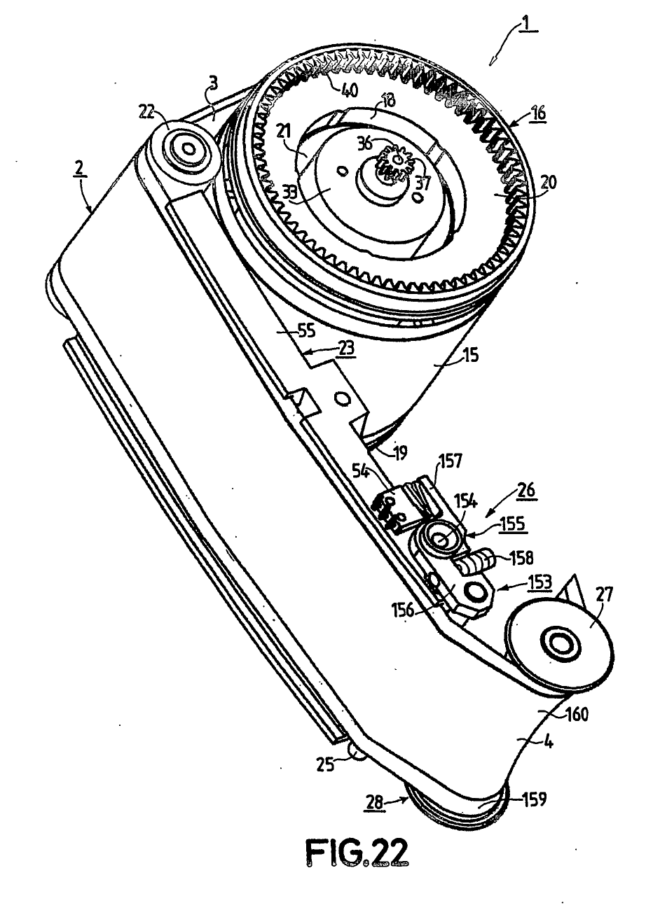

FIG. 22 is an oblique view from above of a further part of the depilation device of FIGS. 1 to 3, this part of the depilation device comprising drive stop means.

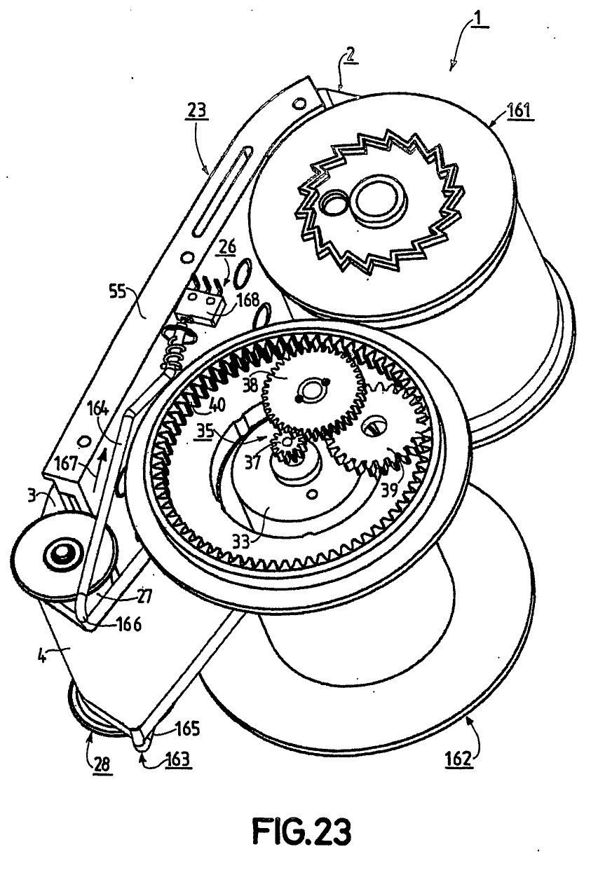

FIG. 23 shows in a similar manner to FIG. 22 a part of a further depilation device, which part of said depilation device also comprises drive stop means.

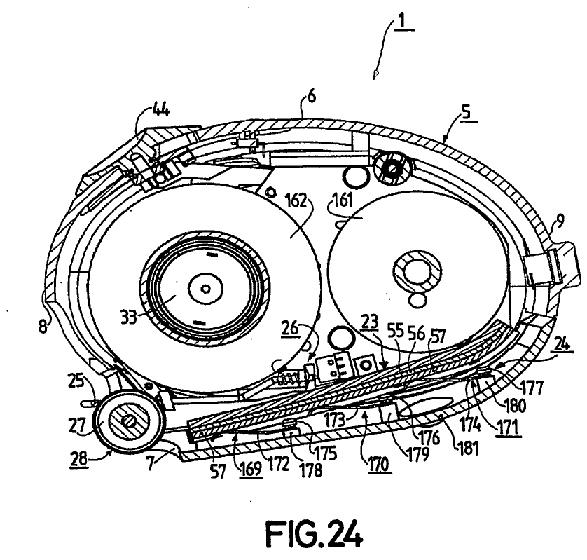

FIG. 24 shows in the same manner as FIG. 3 a further depilation device which comprises a heating device and pressure means.

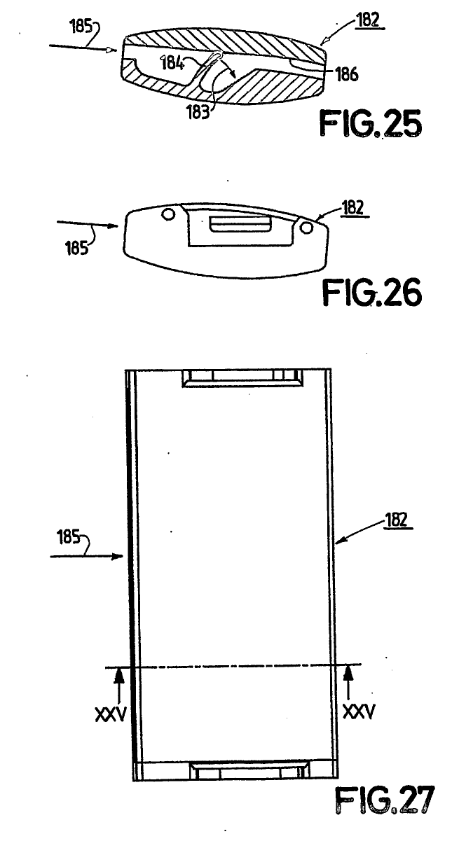

FIGS. 25, 26, and 27 show a stop block of a further depilation device in the same manner as FIGS. 9, 10, and 11.

FIGS. 1, 2, and 3 show a depilation device 1. The depilation device 1 serves to pull hairs from the skin of a person. The depilation device 1 is a so-termed wax depilation device in which the removal of the hairs takes place with the use of wax. In the present case, the wax is not applied to the human skin by means of, for example, a spatula or a similar tool, but by means of a depilation tape 2.

The depilation tape 2 consists essentially of an elongate synthetic resin foil on which wax is provided as a hair removal medium. The depilation tape 2 accordingly has a foil side 3 and a wax side 4. The wax in this case is a wax that can be softened by heating, is applied to the skin of a person after softening, and subsequently cools down, thus enclosing the hairs to be removed, such that subsequently the hairs enclosed by the wax and retained thereby are pulled from the skin in that the depilation tape 2 is pulled off from the skin complete with the cooled-down wax. It is to be noted that alternatively a wax may be used that is suitable for enclosing hairs and pulling out hairs without heating. Both types of wax are commercially available.

The depilation device 1 has a housing 5. The housing 5 in cross-section, as shown in FIG. 3, has a shape of approximately an acute triangle with rounded corners. The housing has a cover region 6, a bottom region 7, a front region 8, a rear region 9, and a first side region 10 and second side region 11. The second side region 1 1 can be taken off the rest of the housing 5, so that it is achieved that the inner region 12 of the depilation device 1 enclosed by the housing 5 is accessible. The depilation tape 2 can thus be easily introduced into the depilation device 1. During operation of the depilation device 1, the depilation device 1 can be moved with its front region 8 in forward direction over the skin of a person in an operational direction 13 indicated by an arrow so as to apply the depilation tape 2 to the skin of the person.

A portion of the inner region 12 of the depilation device 1 is provided and designed as a storage space 14. The storage space 14 is designed for accommodating a store 15 of depilation tape. The store 15 is formed by a tape roll 15 in the present case, which tape roll 15 consists of a plurality of layers of the depilation tape 2 lying on top of one another. The tape roll 15 was formed by winding up of the depilation tape 2.

A reel 16 is provided in the depilation device 1 for holding the tape roll 15 formed by winding up of the depilation tape 2. The reel 16 is accommodated in the storage space 14 and is supported with rotation possibility about a reel axis 17 in the depilation device 1. The reel 16 has a hollow cylindrical sleeve-type hub 18 and two flanges 19 and 20 integrally connected to the hub 18. The flange 19 is visible in FIGS. 20, 21, and 22. The reel 16 is placed with its hub 18 on a bearing sleeve 21, which bearing sleeve 21 is immovably connected to the first side wall region 10. The reel 16 is journaled by means of the bearing sleeve 21.

The reel 16 in the depilation device 1 is designed in a particularly advantageous manner both as a storage reel, i.e. as a supply reel from which the depilation tape 2 can be unreeled for application to the slin of a person, and also as a receiving reel. This means that the reel 16 fulfills a dual function and is provided and constructed not only for unreeling the depilation tape, but also for winding up a depilation tape 2 that was previously applied to the skin of a person. This offers the advantage that no separate winding reel need be provided in the depilation device 1 for receiving the depilation tape 2 that was previously applied to the skin of a person, which is advantageous for obtaining as compact as possible a construction of the depilation device 1. This solution offers the further advantage that depilation tape portions of the depilation tape 2 can be applied several times in succession to the skin of a person and can accordingly be utilized several times in succession for the removal of hairs, which is favorable for minimizing the consumption of depilation tape 2.

It is to be noted that, instead of a reel 16 with a tape roll 15 as a store 15, alternatively a store of depilation tape may be held in a storage space of a depilation device, in which store the depilation tape follows a zigzag or meandering path, and wherein the depilation tape can be driven by a drive drum and a counter-drum pressing the depilation tape against the drive drum, such that the depilation tape is pulled from the storage space by the two drums for being applied to the skin of a person, and is pushed back into the storage space by the two drums during the return of the depilation tape into the storage space.

The depilation tape 2 runs in the inner region 12 of the housing 5 of the depilation device 1 from the storage space 14, i.e. from the reel 16, via a guide roller 22, along a heating device 23 opposite to which contact means 24 are arranged, towards an adjustably mounted tape sensor pin 25 of drive stop means 26, and from the tape sensor pin 25 to an adjustably mounted application roller 27 of application means 28, and from the application roller 27 to a stop block 29 of stop means 30, and finally from the stop block 29 to a free end 31 of the depilation tape 2, an adjustably mounted cutting device 32 being provided adjacent the tape portion between the stop block 29 and the free end 31 of the depilation tape 2.

During a depilation operation, the depilation tape 2 heated by the heating device 23 is applied to the skin by the application roller 27, during which the depilation tape 2 is unreeled from the reel 16 such that the reel 16 is driven, i.e. rotated by the depilation tape 2 being unreeled. It is necessary to drive the reel 16 for winding the depilation tape 2 applied to the skin of a person during such a depilation operation onto the reel 16 again.

To drive the reel 16 for winding up the depilation tape 2 previously applied to the skin of a person, the depilation device 1 has a motor 33. The motor 33 is accommodated in the housing 5 and is constructed for driving the reel 16, as was noted above. The reel 16 in the depilation device 1 has a particularly advantageous construction in which an accommodation space 34 coaxial with the reel axis 17 is provided inside the hollow cylindrical hub of the reel 16. The motor 33 is accommodated in the space 34 such that the reel 16 surrounds the motor 33. The bearing sleeve 21, by means of which the reel 16 is journaled, is present between the motor 33 and the hub 18 of the reel 16, so that the reel 16 is rotatable with respect to the motor 33 inserted into its accommodation space 34. The motor 33 is thus introduced into the hollow cylindrical bearing sleeve 21 immovably supported in the depilation device 1, and the reel 16 is supported with rotation possibility by the bearing sleeve 21. The motor 33 in the depilation device 1 lies entirely within the reel 16, also viewed perpendicularly to the reel axis 17, which is highly advantageous for achieving a particularly space-saving construction.

Between the motor 33 and the reel 16 in the depilation device 1, drive means 35 are provided whose construction is visible in FIGS. 4 and 5. The drive means 35 are constructed as a gear transmission 35. The drive means 35 can be driven by the motor 33, and the drive means 35 can drive the reel 16 for the purpose of winding up the depilation tape 2 that was previously applied to the skin of a person. The gear transmission 35 comprises a first gear 37 fixedly fastened on the motor shaft 36. The gear transmission 35 further comprises a second gear 38 for co-operation with the first gear 37. A third gear situated below the second gear in FIGS. 4 and 5 is coaxially connected to the second gear 38. A fourth, end gear 39 of the gear transmission 35 is in engagement with the third gear. The end gear 39 is in engagement with a toothed rim 40 of the reel 16. The teeth 40 form part of an internal gearing. The end gear 39 is rotatably supported about a spindle 41 immovably provided in the depilation device 1. A carrier 42 is furthermore pivoted about the spindle 41. The second gear 38 and the coaxial third gear are rotatably supported about a spindle 43 on the carrier 42. The second gear 38 and the third gear are held pivoted by means of the carrier 42 such that the second gear 38 can be pivoted between an ON position visible in FIG. 4 and an OFF position visible in FIG. 5. In the ON position, the second gear 38 is in engagement with the first gear 37. In the OFF position, the second gear 38 is out of engagement with the first gear 37. The ON position of the second gear 38 accordingly provides a drive connection between the motor 33 and the reel 16. The OFF position of the second gear 38 breaks the drive connection between the motor 33 and the reel 16 so as to prevent that an unreeling of the depilation tape 2 is hampered by the gear transmission 35.

The depilation device 1 has an ON/OFF slider button 44 for adjusting the carrier 42, which button can be moved between an OFF position visible in FIGS. 3 and 5 and an ON position visible in FIG. 4. The ON/OFF slider button 44 is guided and retained in the cover region of the housing 5 with sliding possibility with respect to the housing 5. The ON/OFF slider button 44 co-operates with a spring (not visible in the figures) which tends to keep the ON/OFF slider button 44 in its OFF position shown in FIGS. 3 and 5. The ON/OFF slider button 44 can be shifted with one finger, preferably the index finger, from its OFF position against the force of the spring connected thereto into its ON position, from which ON position the ON/OFF slider button 44, when released, will automatically return again to its OFF position under the influence of the spring acting thereon.

An adjustment lever 46 pivotable about a fixed shaft 45 is connected to the ON/OFF slider button 44. An intermediate lever 48 is connected to the adjustment lever 46 via a link 47, and is connected again via a further link 49 to the carrier 42. The carrier 42 can be pivoted by means of the adjustment lever 46 and the intermediate lever 48, as is visible in FIGS. 4 and 5. The adjustment lever 46 and the intermediate lever 48 form an adjustment device 50. The adjustment device 50 and the adjustable carrier 42 keep the adjustably supported second gear 38 in engagement with the adjoining first gear 37 of the gear transmission 35 when the ON/OFF slider button 44 is kept in its switching position provided for switching the motor 33 on, i.e. its ON position.

An ON/OFF switch 51 of motor control means 52 of the depilation device 1 can be switched on and off by means of the ON/OFF slider button 44, as is shown diagrammatically in FIG. 6. When the ON/OFF slider button 44 is kept in its ON position, this has the result that the ON/OFF switch 51 assumes its closed state. As is further visible in FIG. 6, the motor control means 52 further comprise a safety switch 53 and a drive stop switch 54. The two switches 53 and 54 are closed in their rest state, as is shown in FIG. 6. In the situation shown in FIG. 6, switching of the ON/OFF switch 51 to its ON position renders it possible to supply to the motor 33 a supply voltage V delivered by a motor supply source (not shown), whereby the motor 33 is switched on. The safety switch 53 is provided and constructed for releasing the motor 33 switch-on. The drive stop switch 54 is provided and constructed for interrupting the power supplied to the motor 33.

It should be noted on the drive means 35 that the drive means 35 need not necessarily be formed by a gear transmission 35. A belt transmission or a friction wheel transmission or a worm wheel transmission may alternatively be used.

The reel 16 in the depilation device 1 thus performs a dual function, because the reel 16 is provided both for unreeling the depilation tape 2 from the tape roll 15 held on the reel 16, so as to apply a portion of the depilation tape 2 to the skin of a person, and for winding the depilation tape 2 onto the tape roll 15 held on the reel 16 so as to pull a portion of the depilation tape 2 previously applied to the skin of a person from the skin of this person again. The reel 16 can be driven into rotation by the drive means 35 here during winding up of the depilation tape 2. During unreeling of the depilation tape 2 from the tape roll 15 of the reel 16, this reel 16 can be driven into rotation by the depilation tape 2 without being hampered by the drive means, i.e. in that the second gear 38 is brought out of engagement with the first gear 37.

As was noted above, the depilation device 1 is provided and constructed for operation with a depilation tape 2 having a hair removal medium that can be softened by heating, which hair removal medium is formed by wax in this case. The depilation device 1 comprises the heating device 23 for heating the depilation tape 2, which device is partly depicted in FIGS. 7 and 8. The depilation tape 2 unreeled from the store 15, i.e. from the tape roll 15 on the reel 16, can be supplied in longitudinal tape direction to the heating device 23, as is visible in FIG. 3. The heating device 23 is capable of heating the tape portion 2A of the depilation tape 2 co-operating at any time with the heating device 23 for the purpose of softening the wax before the depilation tape 2 is applied with its wax side to the skin of a person. The heating device 23 comprises a carrier plate 55. A planar heating element 56 is provided on the carrier plate 55, this element being a resistance heating element. The heating element may be a so-termed PTC heating element. The heating device 23 comprises a planar heating wall 57 at the side of the planar heating element 56 facing away from the carrier plate 55, which wall is formed by an aluminum plate here. The heating element 56 and the heating wall 57 are not shown in FIGS. 7 and 8, but only in FIG. 3. The heating wall 57 is designed for direct co-operation with the depilation tape 2.

The heating device 23 in the depilation device 1 is adjustably mounted between a rest position shown in FIG. 8 and an operational position shown in FIGS. 3 and 7. In its rest position, the heating device 23 is lifted with its heating wall 57 off the depilation tape 2. In its operational position, the heating device 23 has its heating wall 57 in thermally conductive contact with the depilation tape 2, as is visible in FIG. 3. A bearing block 58 is provided in the inner region 12 of the housing 5 so as to enable an adjustment of the heating device 23. Two guide pins 59 and 60 are movably supported in the bearing block 58, which two guide pins 59 and 60 are connected to an additional block 61 and project from the additional block 61. The additional block 61 is integrally formed with the carrier plate 55 of the heating device 23. A return spring (not visible) for the heating device 23 is active between the bearing block 58 and the additional block 61, which return spring tends to keep the heating device 23 in its rest position shown in FIG. 8.

The depilation device 1 has adjustment means 62 for shifting the heating device 23 between its rest position and its operational position. The heating device 23 is kept in its operational position by the adjustment means 62 during unreeling of the depilation tape 2 from the reel 16. The heating device 23 is positioned and retained in its rest position by the return spring during winding up of the depilation tape 2 on the reel 16.

The adjustment means 62 comprise a toggle joint configuration 63 formed by a first toggle joint 64 and a second toggle joint 65, which two toggle joints 64 and 65 are linked together by a link bolt 66. The first toggle joint 64 is linked to the bearing block 58. The second toggle joint 65 is linked to the additional block 61. The adjustment means 62 further comprise a slide 67 which is linked at one side to the link bolt 66 and at the other side via a link 68 (see FIG. 3) to an adjustable retaining bracket 69 which is provided as an adjustable retaining means for the application roller 27. This leads to the situation in the depilation device 1 that the application roller 27 adjustably mounted by means of the retaining bracket 69 forms part of the adjustment means 62. The adjustment of the application roller 27 will be discussed in more detail further below. The action of pressing the application roller 27 against the skin of a person and the resulting adjustment of the application roller 27 in an operational position of the application roller 27 imply that the heating device 23 can be adjusted to its operational position by means of the application roller 27 via the adjustment means 62, as is depicted in FIGS. 3 and 7.

Pressure means 24 are provided in the depilation device 1, as was noted above, which means are arranged opposite the heating device 23 and by which means the tape portion 2A of the depilation tape 2 co-operating with the heating device 23 at any moment is kept in contact with the heating device along a contact zone parallel to the longitudinal tape direction, i.e. along the heating wall 57 of the heating device 23. The contact means 24 in the depilation device 1 have a total of four rows 70, 71, 72, 73 of brackets extending transversely to the longitudinal tape direction of the depilation tape 2, with contact brackets 74, 75, 76, 77 arranged next to one another and shaped as strips. The contact brackets 74 to 77 are arranged opposite the heating wall 57 and keep the tape portion 2A of the depilation tape 2 co-operating at any time with the heating device 23 pressed against the heating wall 57. The contact brackets 74 to 77 are fastened to a support wall 82 of the pressure means 24 by means of respective rivets 78, 79, 80, 81. The contact brackets 74 to 77 are arcuate in shape and are formed from a resilient material, i.e. strip-shaped blade springs. The free ends of the contact brackets remote from the rivets 78 to 81 project into passages 83, 84, 85, 86 in the support wall 82. In an alternative embodiment, the support wall and the contact brackets may be in one piece, for example made from a metal plate of spring steel, in which case the contact brackets are manufactured by stamping and bending. An integral arrangement of synthetic resin is also possible.

When the heating device 23 is kept in its operational position shown in FIGS. 3 and 7, the contact brackets 74 to 77 press practically the entire tape portion 2A against the heating wall 57 of the heating device 23, whereby a particularly good and homogeneous heating of the depilation tape 2 and accordingly of the wax on the depilation tape 2 is achieved.

The depilation tape 2 heated by the heating device 23 together with the heated and thus softened wax is guided over the application roller 27 of the application means 28 during operation for the purpose of applying the depilation tape 2 to the skin of a person. The application means 28 are thus designed for applying the depilation tape 2 taken from the store 15, i.e. unreeled from the tape roll 15, to the skin of this person. The application means 28 comprising the application roller 27 are mounted on carrier means of the depilation device 1, which carrier means are formed by the housing 5 in the present case. This is because the support bracket 69 is pivotably connected to the housing 5 about a pivot axis 87. The support bracket 69 is U-shaped and has two limbs 88 and 89 and a bridge 90 interconnecting the two limbs 88 and 89. The application roller 27 is journaled between the two limbs 88 and 89.

The application roller 27 is adjustably mounted with respect to the housing 5 acting as a carrier means by means of the adjustable support bracket 69, i.e. is pivotably mounted. The application roller 27 is thus adjustable between a rest position visible in FIG. 8 and an operational position visible in FIGS. 3 and 7, i.e. pivotable therebetween. The arrangement is such that the application roller 27, when pressed against the skin of a person, is pivoted from its rest position shown in FIG. 8 into its operational position shown in FIGS. 3 and 7. This pivoting of the application roller 27 and accordingly of the support bracket 69 has the result that the adjustment means 62 connected to the application roller 27 effect an adjustment of the heating device 23. The application roller 27 thus causes the adjustment of an adjustably mounted assembly of the depilation device 1, i.e. the heating device 23. The application roller 27 additionally causes the adjustment of two further adjustably mounted assemblies of the depilation device 1, which will be discussed in more detail further below.

The adjustment means 62 connected to the application roller 27 in the depilation device 1 are not directly connected to the application roller 27, for example to the bearing shaft of the application roller 27, but are connected thereto via the support bracket 69.

In order to apply the depilation tape 2 to the skin of a person, the depilation device 1 is put with the bottom region 7 of its housing 5 on this body part substantially parallel to the body part to be depilated, whereby the application roller 27 is moved from its rest position into its operational position. This adjustment of the application roller 27 leads to the adjustment of the heating device 23 as described above. This adjustment of the application roller 27, however, causes an additional adjustment of an adjustable retaining device for the stop block 29 of the stop means 30, an adjustment of an adjustable blocking device for the reel 16, and an adjustment of a blocking rod for the adjustable cutting device 32. The above measures will be discussed in more detail further below.

As is apparent from FIGS. 1 and 3, the depilation tape 2 is provided with the stop means 30 in the region of the free end 31 remote from the reel 16. The stop means 30 comprise the stop block 29. A channel 91 is provided in the stop block 29, which is shown on a larger scale in FIGS. 9, 10, and 11. The depilation tape 2 is introduced into the channel 91 in an insertion direction 92 (cf. FIGS. 9 to 11) directed towards its free end 31. The depilation tape 2 is held in the channel 91 by a retaining part 93 provided for this purpose. The retaining part 93 is here designed and arranged for clamping the depilation tape 2 in the channel 91. In the embodiment of FIGS. 9 to 11, the retaining part 93 is formed by a cylindrical body 93 which is located in a widened portion 94 of the channel 91 and is loaded by a compression spring 95 such that the cylindrical body 93 is kept pressed against an upper boundary wall 96 of the channel 91. It is achieved thereby that the retaining part 93, i.e. the spring-loaded cylindrical body 93, is designed so as to block an adjustment of the depilation tape 2 with respect to the stop block 29 against the insertion direction 92 and to release an adjustment of the depilation tape 2 with respect to the stop block 29 in the insertion direction 92. The stop block 29 may be shifted against the insertion direction 92 over the depilation tape 2 in a simple manner in that the free end 31 of the depilation tape 2 is held in one hand during such a shifting operation.

To achieve a reliable retention of the stop block 29, the housing 5 has in its bottom region 7 (cf. FIG. 3) an accommodation chamber 97 for accommodating the stop means 30, i.e. the stop block 29 of the stop means 30, in an initial position of the stop block 29. The stop block 29 has two retention steps 98 and 99 for keeping the stop block 29 in the accommodation chamber 97. Each of the two retention steps 98 and 99 is provided in a respective lateral recess 100 and 101 of the stop block 29.

A retention device 102 visible in FIGS. 12 to 17 is provided in the inner region of the housing 5 for retaining the stop block 29. The retention device 102 comprises a bearing block 103. The bearing block 103 has a bottom wall 104 and two side walls 105 and 106. Substantially U-shaped bearing chambers 107 and 108 extend outwards from the side walls 105 and 106. A respective retention lever 111, 112 loaded by a compression spring 109, 110 is pivotably supported about a pivot axis 113, 114 in each of the two bearing chambers 107, 108. The two retention levers 111 and 112 have respective slots 115, 116 adjacent their free ends. The two slots 115 and 116 are provided and constructed for co-operation with the retention steps 98 and 99. A wedge-shaped extension 117, 118 is provided at each of the two retention levers 111, 112. An adjustment pin 119, 120 co-operates with each of the two extensions 117, 118. The two adjustment pins 119 and 120 project from the bridge 90 of the support bracket 69. The extensions 117 and 118 and the adjustment pins 119 and 120 form adjustmeht means for adjusting the retention levers 111 and 112 in dependence on an adjustment of the application roller 27 between its rest position and its operational position. The construction is made such here that, when the application roller 27 is pressed against the skin of a person and is accordingly moved from its rest position shown in FIGS. 12 to 14 into its operational position shown in FIGS. 15 to 17, the two retention levers 111 and 112 are pivoted from their retention position shown in FIGS. 12 to 14 into their release position shown in FIGS. 15 to 17 by means of the adjustment pins 119 and 120 and by means of the wedge-shaped extensions 117 and 118 co-operating with the adjustment pins 119 and 120, with the result that the retention steps 98 and 99 of the stop block are released, so that subsequently the stop block 29 can be moved from the accommodation chamber 97. This is necessary, after the application roller 27 has been applied to the skin of a person such that the depilation tape 2 is pressed against the skin, for making possible a relative movement between the portion of the depilation tape 2 already pressed against the skin of the person, to which the stop block 29 is fastened, and the depilation tape 1 that is moved in the operational direction 13.

Detection means 121 are further provided in the depilation device 1. The detection means 121 are provided and constructed for detecting the presence of the stop means 30, i.e. the stop block 29, in its initial position in the accommodation chamber 97. The detection means 121 for this purpose comprise a sensor part 123 projecting with one end 122 into the accommodation chamber 97. The sensor part 123 is formed by a pin 123 that is supported with longitudinal adjustment possibility. The adjustable support of the pin 123 is realized by means of a bore provided in the bottom wall 104 of the bearing block 103 and by means of a bore in a bearing tag 124 of the bearing block 103. The sensor part 123 is loaded by a compression spring 125 which tends to keep the sensor part 123 in a position such that its free end 122 projects into the accommodation chamber 27. It is achieved thereby that the sensor part 123, when the stop means 30, i.e. the stop block 29 is introduced into its initial position in the accommodation chamber 97, can be moved by the stop block 29 into a release position, which release position is depicted in FIGS. 3, 12, 13, and 14.

The detection means 121 are designed and arranged for co-operating with the motor control means 52. The relevant arrangement is such in the depilation device 1 that the safety switch 53 of the motor control means 52 is arranged adjacent the other end 126 of the sensor part 123, while the sensor part 123 is designed and arranged for co-operating with the safety switch 53 of the motor control means 52. The safety switch 53 is here provided and constructed for releasing the switch-on of the motor 33. The detection means 121, i.e. the sensor part 123 of the detection means 121, co-operate with the motor control means 52, i.e. with the safety switch 53 of the motor control means 52, such that the motor control means 52 release the switch-on of the motor when the detection means detect the presence of the stop block 29 in its initial position in the accommodation chamber 97.

The adjustment of the application roller 27 between its rest position and its operational position in the depilation device 1 is additionally utilized for a further purpose, i.e. in conjunction with the adjustable cutting device 32 of the depilation device 1, as will be explained in more detail below.

First the construction of the adjustable cutting device 32 will be explained with reference to FIGS. 3, 18, 19, 20, and 21. The cutting device 32 has a base wall 127 and two side walls 128 and 129 integral with the base wall 127. The base wall 127 and the two side walls 128 and 129 are arranged such that these three walls 127, 128, 129 form a complementation of the housing 5 of the depilation device 1 when their cutting device 32 is in its rest position shown in FIGS. 3, 18, and 20. In an end region 130 of the base wall 127 remote from the two side walls 128 and 129, a strip-shaped extension 131 projects from the base wall 127, through which extension a bearing shaft 132, rotatably supported in the housing 5, is passed. The cutting device 32 is pivotably supported by means of the bearing shaft 132. An arm 133 projects from the strip-shaped extension 131. Three compression springs 136, each resting on a guide rod 137, are provided between the free end 134 of the arm 133 and a counter block 135 immovably provided in the housing 5. A so-termed dead-center device is realized by means of the counter block 135 and the compression springs 136 and the arm 133, by means of which the cutting device 132 is correctly held both in its rest position and in its operational position.

The cutting device 32 is provided and constructed for cutting the depilation tape 2. The cutting device 32 for this purpose comprises a cutting tool, i.e. an indented knife 138 which extends between the two side walls 128 and 129. A knife with a smooth cutting edge may alternatively be provided.

The cutting device 32 is mounted in the depilation device 1 with a pivoting possibility, i.e. by means of the bearing shaft 132. The cutting device 32 is thus adjustable with respect to the rest of the depilation device 1 between a rest position visible in FIGS. 3, 18, and 19, in which the cutting tool 138 cannot perform a cutting function, and an operational position visible in FIGS. 19 and 21, in which the cutting tool 138 can perform a cutting function. Adjustment means are provided for adjusting the cutting device 32, which means are formed in the present case by the two mutually opposed side walls 128 and 129 of the cutting device 32, which side walls 128 and 129 at the same time form two mutually opposed wall portions of the housing 5 of the depilation device which lie flush with the adjoining housing parts of the housing 5 when the cutting device 32 is in its rest position.

The application means 28 in the depilation device 1, i.e. the application roller 27 is coupled to the cutting device 32 via blocking means 139. The coupling is constructed here such that the cutting device 32 is fixed in its rest position when the depilation tape 2 is being applied to the skin of a person. To safeguard this, the following solution is realized in the depilation device 1 as explained below with reference to FIGS. 18 to 21. The blocking means 139 comprise a blocking rod 140 and a return spring 141 resting on the blocking rod 140. The return spring 141 tends to push the blocking rod 140 towards the support bracket 69 for the application roller 27. The blocking rod 140 abuts with an end 142 facing the application roller 27 against a portion of the bridge 90 of the support bracket 69.

When the application roller 27 is in its operational position as shown in FIGS. 3, 18, and 20, the blocking means 139 coupled to the application roller 27 are kept adjusted against the force of the return spring 141 such that the other end 143 of the blocking rod 140 lies next to a checking surface 144, which checking surface 144 is provided in the region of the strip-shaped extension 131. It is achieved thereby that pivoting of the cutting device 32 from its rest position into its operational position is inhibited.

In the area of the strip-shaped extension 131, the cutting device 32 is provided with an arm 145 projecting from the extension 131. A slotted hole 145A is provided in the arm 145. A pin 146 of an angled lever 147 projects into the slotted hole 145A, which angled lever 147 is provided as a blocking device for the reel 16. The angled lever 147 pivots about a shaft 148. The pin 146 is provided in the region of the free end of a first arm 149 of the angled lever 147. In the region of the free end of a second arm 150 of the angled lever 147, a blocking pin 151 is provided so as to project from the second arm 150. The blocking pin 151 is designed for co-operating with the teeth 40 of the reel 16. When the application roller 27 is in its operational position as shown in FIGS. 18 and 20, the blocking pin 151 is out of engagement with the teeth 140. The reel 116 can thus be driven in this case so as to apply depilation tape 2 to the skin of a person, while at the same time the cutting device 32 is blocked in its rest position, so that any injuries caused by the cutting tool 138 are prevented.

When the depilation device 1 is lifted off the skin again with its application roller 27 after an application of depilation tape 2 to the skin of a person, the application roller 27 will return into its rest position shown in FIGS. 19 and 21 under the influence of the return spring (not shown) for the heating device 23. This has the result that the blocking rod 140 is moved in the direction of the application roller 27, whereby the other end 143 of the blocking rod 140 is moved away from the checking surface 144. This again has the result that the other end 131 of the blocking rod 140 is no longer in the adjustment path of the checking surface 144. When a user of the depilation device 1 in this operational state pivots the cutting device 32 by means of the two side walls 128 and 129 into its operational, i.e. cutting position visible in FIGS. 16 and 18, this will have the result that the angled lever 147 is adjusted by means of the slotted hole 145 and the pin 146 so far that the blocking pin 151 comes into engagement with the teeth 40 of the reel 16 and thus prevents a rotary drive of the reel 16, so that an undesired unreeling of the depilation tape 2 from the reel 16 is prevented during cutting of the depilation tape 2. Furthermore, a further checking surface 152 of the cutting device 32 extending in a circular arc is positioned opposite the other end 143 of the blocking rod 140 when the cutting device 32 is in its operational position, i.e. when the cutting device 32 is open, which achieves that the blocking rod 140 cannot be shifted in the direction of the bearing shaft 132 anymore. The blocking rod 140 is thus blocked against shifting, which again has the result that the application roller 27 is locked in its rest position and can accordingly not be pivoted from its rest position into its operational position. The latter is important because the reel 16 is blocked against rotation during cutting of the depilation tape 2, and accordingly the depilation tape 2 is tensioned, which would lead to an adjustment of the application roller 27 and accordingly a release of the stop block 29, but this is prevented in that the application roller 27 is locked. A further safety switch is provided in the depilation device 1, but this is not shown in the drawings. The further safety switch is arranged such that said switch is opened when the cutting device 32 is pivoted into its operational position, whereby the power circuit of the motor 33 is interrupted.

The depilation device 1 is connected to a public power mains for normal operation of the depilation device 1, which has the result that the heating device 23 is provided with energy and thus heats the heating wall 57. A certain waiting time is to be observed here until the heating wall 57 has reached a sufficiently high temperature for achieving a satisfactory heating of the depilation tape 2. The moment the depilation tape 2 can be sufficiently heated, the person using the depilation device 1 applies the application roller 27 to the body part to be depilated, which will have the results that the application roller 27 is pivoted into its operational position and accordingly the heating device 23 is moved towards the depilation tape 2, that the retention device 102 releases the stop block 29 so that the stop block 29 is pressed from the accommodation chamber 29 in conjunction with the detection means 121 and the safety switch 53 of the motor control means 52 is opened by the detection means 121, and that the cutting device 32 is locked against opening. Subsequently, the depilation device 1 is moved in the operational direction 13 over the body part to be depilated, during which the depilation tape will adhere to this body part, with the result that the stop block 29 is retained by the depilation tape 2 in a starting position on the skin of the person using the depilation device 1. When the application roller 27 has reached the end of the body part to be depilated, the person using the depilation device 1 lifts the application roller 27 from the skin, with the results that the heating device 23 and the application roller 27 are automatically returned from their operational positions to their rest positions by means of the return spring for the heating device 23, that the retention levers 111 and 112 provided for retaining the stop block 29 return to their positions suitable for holding the stop block 29, and that the cutting device 32 is released.

Subsequently, the depilation device is returned to the starting point of the depilation process against the operational direction 13 accompanied by a further unreeling of depilation tape 2 from the reel 16, i.e. so far that the depilation device 1 can be applied with its accommodation chamber 97 on the stop block 29. Then the accommodation chamber 97 is moved over the stop block 29 and the depilation device 1 is pressed with its bottom region 7 against the skin, with the result that the stop block 29 enters the accommodation chamber 97 and thus operates the detection means 121. After the stop block 29 has been fully inserted into the accommodation chamber 97, the stop block 29 is held by the retention means 102 and is fixed in the accommodation chamber 97. The adjustment of the detection means 121 operates the safety switch 53, so that this safety switch 53 is switched to its conductive state. In this operational situation, a portion of the depilation tape 2 lies against the skin of a person, which portion extends from the stop block 29 up to an application end point, while an approximately equally long portion extends from the application end point back to the depilation device 1.

Subsequently, the person using the depilation device 1 will allow a certain waiting time to pass, during which waiting time the previously heated wax cools down and during this encloses the hairs to be removed. The person using the depilation device 1 must now shift the ON/OFF slider button 44 to its ON position in order to pull the portion of the depilation tape 2 applied to the skin from the skin again. This establishes the drive connection between the motor 33 and the reel 16 by means of the second gear 38 of the drive means 35, and the ON/OFF switch 51 is closed, i.e. brought into its conductive state. The result of this is that the motor 33 is provided with energy and accordingly drives the reel 16. This drives the depilation tape 2 and accordingly pulls it off the skin of the person. The drive of the motor 33 persists so long until the depilation tape 2 is tensioned between the stop block 29 held by the retention means 102 and the tape roll 15 on the reel 16. The moment the depilation tape 2 starts being tensioned, the drive stop means 26 are activated, the construction of which will be explained in more detail below with reference to FIG. 22.

The drive stop means 26 are provided and constructed for automatically ending the drive of the reel 16. The drive stop means 26 automatically end the drive of the reel 16 upon reaching the end of the winding process of the depilation tape 2 previously applied to the skin of a person. The drive stop means 26 in the depilation device 1 are constructed for automatically switching off the motor 33. The drive stop means 26 comprise sensor means 153, which sensor means 153 are supported with adjustment possibility between a rest position and a switching position. The sensor means 153 comprise a support part 155 pivoting about a pivot axis 154, which part is constructed as a two-arm lever with a first lever arm 156 and a second lever arm 157. A compression spring 158 acts on the first lever arm 156, which compression spring 158 forms spring means by which the sensor means 153 are biased in the direction of their rest position shown in FIG. 22. The sensor means 153 further comprise the tape sensor pin 25, which tape sensor pin 25 is supported by the support part 155, i.e. by the first support arm 156 of the support part 155, and which projects from the support part 155. The tape sensor pin 25 forms a sensor portion of the sensor means 153 and is designed and arranged for co-operating with the depilation tape 2. The length of the tape sensor pin 25 is greater than the width of the depilation tape 2, which is favorable for a smooth co-operation. The tape sensor pin 25 projects into the path of movement of the depilation tape 2 traversed by the depilation tape 2 during winding of this depilation tape 2 onto the reel 16. When the end of the winding process of the depilation tape 2 previously applied to the skin of a person has been reached, i.e. when the tensioning of the depilation tape 2 mentioned above starts, the sensor means 153 trigger the automatic ending of the drive of the reel 16. The drive stop switch 54 is provided for this purpose. The drive stop switch 54 is designed and arranged for co-operating with the sensor means 153 such that the second lever arm 157 of the support part 155 is capable of operating, i.e. switching the drive stop switch 54. A movement of the sensor means 153 from the rest position shown in FIG. 22 into the switching position not shown in FIG. 22 against the force of the compression spring 158 owing to the displacement of the tape sensor pin 25 caused by the tensioned depilation tape 2 will have the result that the drive stop switch 54 is operated by the second lever arm 157, again with the result that the drive stop switch 54 is switched to its non-conductive state. The motor 33 is switched off thereby and remains switched off as long as the depilation tape 2 is kept under tension.

It is to be noted on FIG. 22 that it is more clearly visible in FIG. 22 that the depilation tape 2 is formed by a foil 159 and a wax layer 160 provided on the foil 159. FIG. 23 shows a portion of a further depilation device 1. A storage reel 161 and a winding reel 162 are provided in the depilation device 1 of FIG. 23. The winding reel 162 can be driven by a motor 33 accommodated in the winding reel 162 in the same manner as in the depilation device 1 of FIGS. 1 to 22, i.e. also via a gear transmission 35.

A heating device 23 is also provided in the depilation device 1 of FIG. 23, serving to heat the depilation tape 2. In this case the heating device 23 is immovably supported in the depilation device 1. This is because a previously heated portion of the depilation tape 2 that was applied to the skin of a person is not transported back along the heating device 23 to the storage reel 161, but is wound up on the winding reel 162.

Drive stop means 26 are again provided in the depilation device 1 of FIG. 23. The drive stop means 26 in this case comprise a substantially U-shaped wire bracket 163 which has two limb portions 164 and 165 and a bridge portion 166 interconnecting the two limb portions 164 and 165. The wire bracket 163 is supported with sliding possibility by guide means (not shown) parallel to the arrow 167 shown in FIG. 23. A drive stop switch 168 is provided for co-operating with the wire bracket 163, i.e. with the first limb portion 164 of the wire bracket 163, which switch corresponds to the drive stop switch 54 of the depilation device 1 of FIGS. 1 to 22 in its manner of operation. When the end of a winding process for the depilation tape 2 has been reached in the depilation device 1 of FIG. 23, the wire bracket 163 is shifted in the direction of the arrow 167 towards the drive stop switch 168, which operates the switch and thus leads to a switch-off of the motor 33.

FIG. 24 shows a further depilation device 1. In this depilation device 1, again, a storage reel 161 and a winding reel 162 are provided, and the winding reel 162 can be driven by a motor 33.

In the depilation device 1 of FIG. 24, a heating device 23 immovably supported in the depilation device 1 is provided again, as was the case in the depilation device 1 of FIG. 23. Pressure means 24 are also provided in the depilation device 1 of FIG. 24, by which means a portion of the depilation tape 2 extending in the longitudinal tape direction is pressed against the heating device 23, i.e. against the heating wall 57 of the heating device 23. Three rows 169, 170, and 171 of brackets extending transversely to the longitudinal tape direction are provided in this case, four contact brackets 172, 173, 174 being provided in each row of brackets 169, 170, 171. The contact brackets 172, 173, 174 are constructed as wires in this case and are formed from a spring wire material. The contact brackets 172, 173, 174 are fastened to a planar support wall by means of fastening screws 175, 176, 177 which are screwed into fastening blocks 178, 179, 180, said support wall being formed by a housing wall 181 of the housing 5 of the depilation device 1 in this case.

FIGS. 25, 26, and 27 show a stop block 182. The stop block 182 differs from the stop block 29 of FIGS. 9, 10, and 11 in that a movable clamping tag 184 is provided, i.e. a tag pivotable in the direction of an arrow 183, instead of a cylindrical body 93 loaded by a spring 95. A depilation tape can be introduced into the stop block 182 in the direction of an arrow 185 without major difficulties also in the construction of the stop block 182, i.e. be passed through the channel 186 in the stop block 182, whereas a removal of the depilation tape from the stop block 182 against the direction of the arrow 185 is prevented by the clamping tag 184. The stop block 182 can also be easily shifted against the direction of the arrow 185 over the depilation tape passed through the stop block 182.

1. A depilation device (1) comprising a housing (5) and a reel (16) said reel (16) being supported in the depilation device (1) so as to be rotatable about a reel axis (17) and being provided and constructed for accommodating a tape roll (15) formed by winding up of a depilation tape (2), wherein the reel (16) performs a dual function and is designed both for unreeling the depilation tape (2) from the tape roll (15) present on the reel (16) so as to apply a portion of the depilation tape (2) to the skin of a person, and also for winding up the depilation tape (2) onto the tape roll (15) present on the reel (16) so as to pull off a portion of the depilation tape (2) previously applied to the skin of a person from the skin of this person again, and wherein the reel (16) can be driven into rotation by drive means (35) during winding up of the depilation tape (2), and wherein the reel (16) can be driven into rotation by the depilation tape (2) during unreeling of the depilation tape (2) without being hampered by the drive means (35).

2. A depilation device (1) as claimed in claim 1, wherein the drive means (35) comprise an electric motor (33).

3. A depilation device (1) as claimed in claim 2,
wherein the electric motor (33) comprises a motor shaft (36),
wherein a first gear (37) is fixedly mounted on the motor shaft (36),
wherein the drive means (35) are formed by a gear transmission (35) comprising the first gear (37), which gear transmission (35) comprises an end gear (39) which is in engagement with a toothed rim (40) of the reel (16), and
wherein one gear (38) of the gears (37, 38, 39) of the gear transmission (35) is adjustably supported by an adjustable carrier (42) and can be brought thereby out of engagement with an adjoining gear (37) of the gear transmission (35) so as to avoid that the unreeling of the depilation tape (2) is hampered by the drive means (35).

4. A depilation device (1) as claimed in claim 1,
wherein the depilation device (1) is provided and constructed for operation with a depilation tape (2) comprising a hair removal medium that can be softened by heating,
wherein the depilation device (1) comprises a heating device (23) to which the depilation tape (2) unreeled from the reel (16) can be supplied and by means of which the relevant tape portion (2A) of the depilation tape (2) co-operating with the heating device (23) can be heated for the purpose of softening the hair removal medium before the depilation tape (2) is applied with its hair removal medium to the skin of a person,
wherein the heating device (23) is mounted with adjustment possibility between a rest position and an operational position, is lifted off the depilation tape (2) in its rest position, and is in thermally conductive contact with the depilation tape (2) in its operational position, and wherein adjustment means (62) are provided for adjusting the heating device (23), said means being capable of positioning the heating device (23) in its operational position during unreeling of the depilation tape (2) from the reel (16) and of positioning the heating device (23) in its rest position during winding up of the depilation tape (2) onto the reel (16).

5. A depilation device (1) as claimed in claim 4,
wherein the depilation device (1) comprises an application roller (27) for applying the depilation tape (2) to the skin of a person, which application roller (27) can be pressed against the skin of a person with the depilation tape (2) being interposed between them,
wherein said application roller (27) is adjustably mounted and forms part of the adjustment means (62), and
wherein the heating device (23) can be adjusted into its operational position by means of the application roller (27) via the adjustment means (62) in that the application roller (27) is pressed against the skin, whereby the application roller (27) is held in its adjusted position.
